(19) United States
(12) Patent Application Publication
(10) Pub. No.: US 2026/0076711 A1
(43) Pub. Date: Mar. 19, 2026

(54) BIOPSY NEEDLE POSITIONING SYSTEM, BIOPSY NEEDLE POSITIONING METHOD, AND MEDICAL DEVICE SYSTEM

(71) Applicant: GE Precision Healthcare LLC, Waukesha, WI (US)

(72) Inventors: Ziqiang Du, Wuxi (CN); Feng Li, Beijing (CN); Xinyuan Wang, Beijing (CN)

(21) Appl. No.: 19/332,817

(22) Filed: Sep. 18, 2025

(30) Foreign Application Priority Data
Sep. 18, 2024 (CN) 202411298803.X

(51) Int. Cl. A61B 17/34; A61B 10/02; A61B 90/00

(52) U.S. Cl. CPC A61B 17/3403; A61B 10/0233; A61B 90/361; A61B 90/39

(57) ABSTRACT

A biopsy needle positioning system, a biopsy needle positioning method, and a medical device system is described. The biopsy needle positioning system includes a marker assembly including a marker and a cannula that is fixed relative to the marker and carries a biopsy needle, an image acquisition apparatus being mounted at a position at which the marker can be photographed, and configured to acquire an RGB image of the marker and a depth image corresponding to the RGB image, and a positioning apparatus, which extracts an RGB feature and a point cloud feature of the marker based on the RGB image and the depth image of the marker by using a trained network model, and estimates an estimated position and attitude of the marker based on the RGB feature and the point cloud feature, to obtain an insertion point and an insertion angle of the biopsy needle.

### CROSS-REFERENCE TO RELATED APPLICATIONS

This application claims priority to Chinese Application No. 202411298803.X, filed on Sep. 18, 2024, the disclosure of which is incorporated herein by reference in its entirety.

### TECHNICAL FIELD

The present application relates to the field of medical imaging, and in particular, to a biopsy needle positioning system, a biopsy needle positioning method, and a medical device system.

### BACKGROUND

A biopsy is the removal of cells, tissues or fluids from a patient for examination, to determine whether the patient has a disease or whether a disease which the patient suffers from lead to a lesion. Generally, a biopsy needs to be supported by an additional device, such as a robotic arm or a simple auxiliary clamp. The deployment space of a robotic arm is large, and precise insertion points and angles can be automatically provided without relying on an operator. Compared with a robotic arm, a simple auxiliary clamp helps to affix a biopsy needle, but each operation must be performed manually.

Biopsies have important applications in pathological diagnosis, and biopsies can assist in clinically diagnosing lesions or providing clues for disease diagnosis, understanding the nature and development trend of lesions, and providing a reference basis for clinical medication.

It should be noted that the above introduction of the background is only for the convenience of clearly and completely describing the technical solutions of the present application, and for the convenience of understanding for those skilled in the art. The above technical solutions are not considered to be well known to those skilled in the art merely because they are set forth in the Background of the present application.

### SUMMARY OF THE INVENTION

The inventor has found that, in actual operation, although a robotic arm can provide precise insertion points and angles, robotic arms are expensive, and have a large deployment space. When an insertion point and an insertion angle of a biopsy needle are changed, the robotic arm must be re-calibrated and aligned, which wastes a lot of time. In addition, the use of a simple auxiliary clamp is largely dependent on the experience of an operator, and an inexperienced operator may cause an incorrect insertion point and angle, which reduces work efficiency and increases the pain of a patient.

In order to solve at least one of the above problems or other similar problems, embodiments of the present application provide a biopsy needle positioning system, a biopsy needle positioning method, and a medical device system. A marker is fixed on a cannula carrying a biopsy needle, a real position and attitude of the marker are estimated based on an acquired RGB image corresponding to the marker and an acquired depth image corresponding to the RGB image, and an estimated position and attitude of the marker are obtained by using a network model, to obtain an insertion point and angle of the biopsy needle. Thus, biopsy costs can be reduced, and work efficiency can be improved.

According to an aspect of the embodiments of the present application, provided is a biopsy needle positioning system. The system includes a marker assembly, the marker assembly comprising a marker, and a cannula that is fixed relative to the marker and carries a biopsy needle; an image acquisition apparatus, the image acquisition apparatus being mounted at a position at which the marker can be photographed, and configured to acquire an RGB image of the marker and a depth image corresponding to the RGB image; and a positioning apparatus, which extracts an RGB feature and a point cloud feature of the marker based on the RGB image and the depth image of the marker by using a trained network model, and estimates an estimated position and attitude of the marker based on the RGB feature and the point cloud feature, to obtain an insertion point and an insertion angle of the biopsy needle.

In some embodiments, the marker assembly further comprises a bracket connected to one end of the cannula, and the marker is mounted on the bracket. In some embodiments, the bracket is L-shaped. In some embodiments, the marker is a cube, a sphere, or a cone. In some embodiments, the positioning apparatus segments the RGB image by using a segmentation network, to separate a marker portion from the RGB image; extracts an RGB feature of the marker portion, by using the marker portion as an input to an RGB network; extracts a point cloud feature of a point cloud portion corresponding to the marker portion on the depth image, by using the point cloud portion as an input to a point cloud network; and obtains a real position and attitude of the marker based on the RGB feature and the point cloud feature.

In some embodiments, the positioning apparatus further projects the depth image into two dimensions, samples obtained two-dimensional points, and predicts a boundary of the marker by using a boundary prediction network; and the positioning apparatus obtains the real position and attitude of the marker based on the RGB feature, the point cloud feature, and the boundary of the marker.

In some embodiments, the system further includes a training apparatus, configured to train the network model used by the positioning apparatus by using a plurality of RGB images of the marker at different angles and depth images corresponding to the RGB images, that are acquired by the image acquisition apparatus, and real positions and attitudes of the marker at the different angles.

In some embodiments, for each of the RGB images and depth images corresponding to the RGB images, the training apparatus converts coordinates P_{world} of a plurality of marking points on the marker in a world coordinate system into coordinates P_{camera} in a camera coordinate system, and converts the coordinates P_{camera} of the plurality of marking points in the camera coordinate system into coordinates P_{ct} in a CT coordinate system, to obtain an estimated position and attitude of the marker at a current angle.

In some embodiments, the training apparatus converts the coordinates P_{world} of the plurality of marking points in the world coordinate system into coordinates P_{camera} in the camera coordinate system according to the following formula:

$${P}_{camera} = {R}_{wc} \cdot {P}_{world} + {t}_{wc}$$

wherein R_{we} and t_{wc} are external parameters of the image acquisition apparatus, R_{wc} being a rotation matrix and t_{wc} being a translation vector, which represent a position and a direction of the camera coordinate system relative to the world coordinate system, respectively.

In some embodiments, the training apparatus converts the coordinates P_{camera} of the plurality of marking points in the camera coordinate system into coordinates P_{ct} in the CT coordinate system according to the following formula:

$${P}_{ct} = {R}_{ct} \cdot {P}_{camera} + {t}_{ct}$$

wherein R_{ct} is a rotation matrix and t_{ct} is a translation vector, which represent a position and a direction of the CT coordinate system relative to the camera coordinate system, respectively, R_{ct} and t_{ct} being calculated by means of a singular value decomposition method.

In some embodiments, the plurality of marking points are located at different positions of the marker, and the plurality of marking points represent a contour of the marker. In some embodiments, the system further includes a display apparatus, the display apparatus displaying an insertion point and an insertion angle of the biopsy needle.

According to another aspect of the embodiments of the present application, provided is a biopsy needle positioning method. The method includes acquiring an RGB image of a marker and a depth image corresponding to the RGB image, wherein the marker is connected and fixed to a cannula that is fixed relative to the marker and carries a biopsy needle; extracting an RGB feature and a point cloud feature of the marker based on the RGB image and the depth image of the marker, by using a trained network model; and estimating an estimated position and attitude of the marker based on the RGB feature and the point cloud feature, to obtain an insertion point and angle of the biopsy needle.

According to yet another aspect of the embodiments of the present application, provided is a medical device system. The medical device system includes the biopsy needle positioning system according to an embodiment of the first aspect.

One of beneficial effects of the embodiments of the present application lies in that the marker is fixed on the cannula carrying the biopsy needle, the real position and attitude of the marker are estimated based on the acquired RGB image corresponding to the marker and the depth image corresponding to the RGB image, and the estimated position and attitude of the marker are obtained by using the network model. Thus, the insertion point and angle of the biopsy needle are calculated by using the position and the attitude of the marker, the position of which is fixed relative to the biopsy needle, and the biopsy needle can be accurately positioned in real time without the need for a robotic arm and a re-calibration process, thereby reducing biopsy costs and improving work efficiency.

With reference to the following description and drawings, specific implementations of the present application are disclosed in detail. It should be understood that the implementations of the present application are not limited in scope thereby. Within the scope of the spirit and clauses of the appended claims, the implementations of the present application comprise many changes, modifications, and equivalents.

The features described and/or illustrated for one implementation may be used in one or more other implementations in the same or similar manner, be combined with features in other embodiments, or replace features in other implementations.

It should be emphasized that the term “include/comprise/have”, when used herein, refers to the presence of features, integrated components, or assemblies, but does not preclude the presence or addition of one or more other features, integrated components, or assemblies.

### BRIEF DESCRIPTION OF THE DRAWINGS

The above and other objects, features and advantages of the embodiments of the present application will become more apparent from the following detailed description with reference to the drawings, in which:

FIG. 1 is a schematic diagram of a biopsy needle positioning system according to an embodiment of the present application.

FIG. 2 is a schematic diagram of a cannula according to an embodiment of the present application.

FIG. 3 is a schematic diagram of a marker according to an embodiment of the present application.

FIG. 4 is a schematic diagram of a marker assembly according to an embodiment of the present application.

FIG. 5 is a schematic diagram of a working process of a positioning apparatus according to an embodiment of the present application.

FIG. 6 is a schematic diagram of a working process of a trained network model according to an embodiment of the present application.

FIG. 7 is a schematic diagram of an action process of a training apparatus according to an embodiment of the present application.

FIG. 8 is a schematic diagram of conversion from a world coordinate system to a camera coordinate system.

FIG. 9 is a schematic diagram of a biopsy needle positioning method according to an embodiment of the present application.

FIG. 10 is a schematic diagram of a CT imaging device according to an embodiment of the present application.

FIG. 11 is a schematic diagram of a CT imaging system according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the present application may be employed are indicated. It should be understood that the present application is not limited to the described implementations, and include all modifications, variations, and equivalents which fall within the scope of the appended claims.

In the embodiments of the present application, the terms “first”, “second”, “upper”, “lower”, etc. are used to distinguish different elements with respect to naming, but do not represent a spatial arrangement, a temporal order, etc. of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one embodiment may be used in one or more other embodiments in an identical or similar manner, combined with features in other embodiments, or replace features in other embodiments.

Embodiments of the present application provide a biopsy needle positioning system.

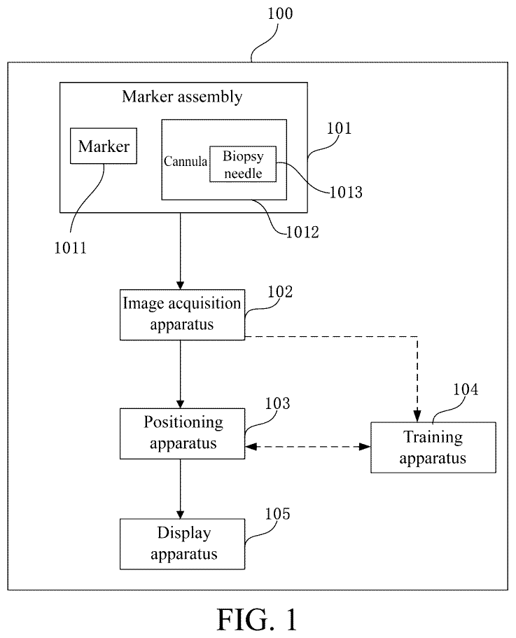

FIG. 1 is a schematic diagram of a biopsy needle positioning system according to an embodiment of the present application. As shown in FIG. 1, the biopsy needle positioning system 100 includes: a marker assembly 101, the marker assembly 101 including a marker 1011 and a cannula 1012, the cannula 1012 carrying a biopsy needle 1013, and the cannula 1012 being fixed relative to the marker 1011; an image acquisition apparatus 102, the image acquisition apparatus 102 being mounted at a position at which the marker 1011 can be photographed, and configured to acquire an RGB image of the marker 1011, and a depth image corresponding to the RGB image; and a positioning apparatus 103, the positioning apparatus 103 extracting an RGB feature and a point cloud feature of the marker 1011 based on the RGB image and the depth image of the marker 1011, by using a trained network model, and estimating an estimated position and attitude of the marker 1011 based on the RGB feature and the point cloud feature, to obtain an insertion point and an insertion angle of the biopsy needle.

According to the described embodiment, the marker is fixed on the cannula carrying the biopsy needle, a real position and attitude of the marker are estimated based on the acquired RGB image corresponding to the marker and the depth image corresponding to the RGB image, and the estimated position and attitude of the marker are obtained by using the network model. Thus, the insertion point and angle of the biopsy needle are calculated by using the position and the attitude of the marker, the position of which is fixed relative to the biopsy needle, and the biopsy needle can be accurately positioned in real time without the need for a robotic arm and a re-calibration process, thereby reducing biopsy costs and improving work efficiency. The marker assembly in this embodiment of the present application is described below.

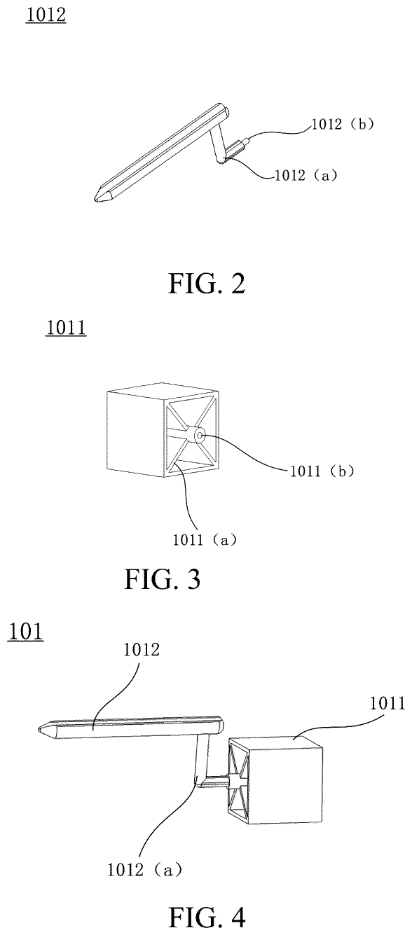

FIG. 4 is a schematic diagram of a marker assembly according to an embodiment of the present application. As shown in FIG. 4, the marker assembly 101 includes the marker 1011 and the cannula 1012. FIG. 2 is a schematic diagram of a cannula according to an embodiment of the present application.

In some embodiments, as shown in FIG. 2 and FIG. 4, the marker assembly 101 includes the cannula 1012, the cannula 1012 is configured to accommodate a biopsy needle (not shown in the figures), and the biopsy needle can be firmly placed in the cannula 1012, that is, a relative position between the cannula 1012 and the biopsy needle is fixed, so as to facilitate a subsequent movement operation and acquisition of a position and an attitude of the biopsy needle.

In the described embodiment, the material and shape of the cannula 1012 and the type of the biopsy needle are not limited, as long as the cannula 1012 can accommodate and fix different types of biopsy needles, and the biopsy needle can perform a biopsy.

In some embodiments, the cannula 1012 has a bracket for placing the marker 1011. As shown in FIG. 2 and FIG. 4, the cannula 1012 has a bracket 1012(a) at a head region, and the bracket 1012(a) is configured to fix the marker 1011. In the embodiments of FIG. 2 and FIG. 4, an L-shaped bracket 1012(a) is used as an example. The structure of the bracket is not limited in the present application, as long as the bracket can be configured to fix the marker 1011, so that the marker 1011 is fixed relative to the cannula 1012.

FIG. 3 is a schematic diagram of a marker according to an embodiment of the present application. As shown in FIG. 3 and FIG. 4, the marker assembly 101 includes the marker 1011.

In at least one example, as shown in FIG. 3, the marker 1011 is a cube having a side length of 40 mm, and the cube has a support frame 1011(a) to ensure that the shape of the marker 1011 is not deformed by extrusion, thereby further improving the accuracy of determining a position and an attitude of the marker 1011. The side length of the marker 1011 may alternatively be other lengths, as long as they can facilitate insertion of the biopsy needle and calculation of the position and the attitude of the marker 1011.

The marker 1011 may alternatively be other shapes, such as a symmetrical sphere or cone. When the marker 1011 is a three-dimensional symmetrical figure, only a part of the position and the attitude of the marker 1011 needs to be calculated to obtain the position and the attitude of the entire marker 1011, thereby obtaining the insertion point and angle of the biopsy needle. The marker 1011 may alternatively not be a three-dimensional symmetrical figure, and in this case, the position and the attitude of the marker 1011 need to be calculated for the entire marker.

In the described embodiment, the surface of the marker 1011 may be covered with black, which helps block reflection of light, so as to be captured by a camera, thereby improving the accuracy of photographing by the image acquisition apparatus 102.

In the described embodiment, the marker 1011 and the cannula 1012 of the marker assembly 101 may be fixed by means such as fitting or snapping. Taking FIG. 2 and FIG. 3 as an example, the cannula 1012 has a protruding part 1012(b), the marker 1011 has an insertion part 1011(b), and the protruding part 1012(b) is inserted into the insertion part 1011(b), so that the marker 1011 is fixed relative to the cannula 1012, to form the marker assembly 101 shown in FIG. 4. In addition, the cannula 1012 and the marker 1011 may alternatively be integrally formed, or may be fixed by other means.

As shown in FIG. 4, the marker 1011 is fixed at a tail part of the cannula 1012, and the marker 1011 may alternatively be fixed at other parts of the cannula 1012, as long as the image acquisition apparatus 102 can photograph the marker, and insertion of the biopsy needle is not affected.

According to the described embodiment, by means of fixing the marker that can be captured by the image acquisition apparatus on the cannula, the position and the attitude of the biopsy needle can be calculated by means of estimating the position and the attitude of the marker, to obtain the insertion point and angle of the biopsy needle.

In some embodiments, the image acquisition apparatus 102 is used to acquire the RGB image of the marker 1011 and the depth image corresponding to the RGB image. The image acquisition apparatus 102 can acquire views of a plurality of markers 1011 from different angles. The image acquisition apparatus may be an RGB-D camera, such as a D345 camera, or may be another camera capable of capturing the RGB image, and the depth image corresponding to the RGB image.

In the described embodiment, the image acquisition apparatus 102 is mounted at a position at which the marker 1011 can be photographed, for example, a position at an opposite side of an operator and 50 cm away from the operator's hand, or may be mounted at other positions, as long as the marker 1011 can be photographed.

In some embodiments, after the image acquisition apparatus 102 acquires the RGB image and the depth image of the marker 1011, the positioning apparatus 103 can estimate the position and the attitude of the marker 1011 based on the RGB image and the depth image of the marker 1011, by using a trained network. The trained network herein includes a segmentation network, an RGB network, and a point cloud network. The following describes an action of the positioning apparatus 103 in this embodiment of the present application, with reference to the accompanying drawings.

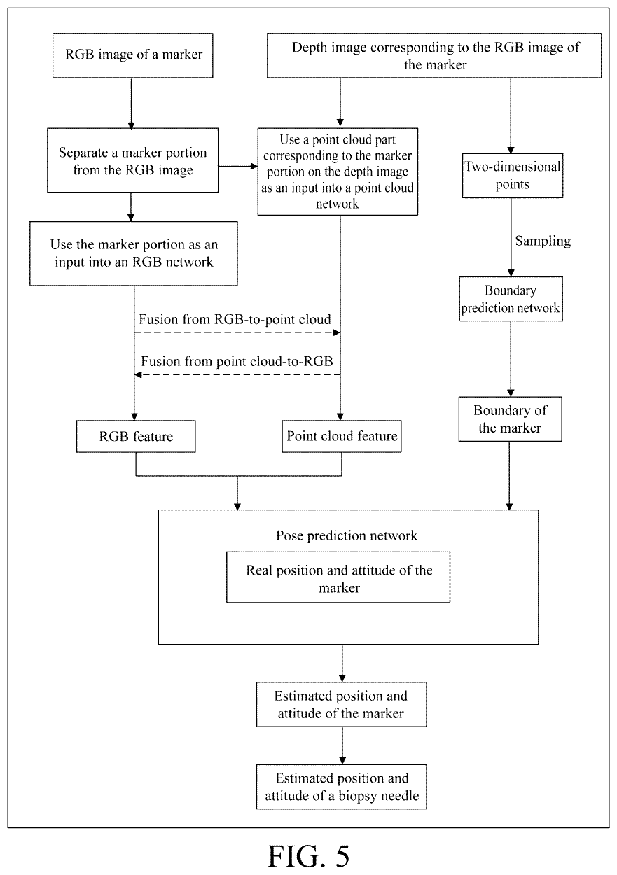

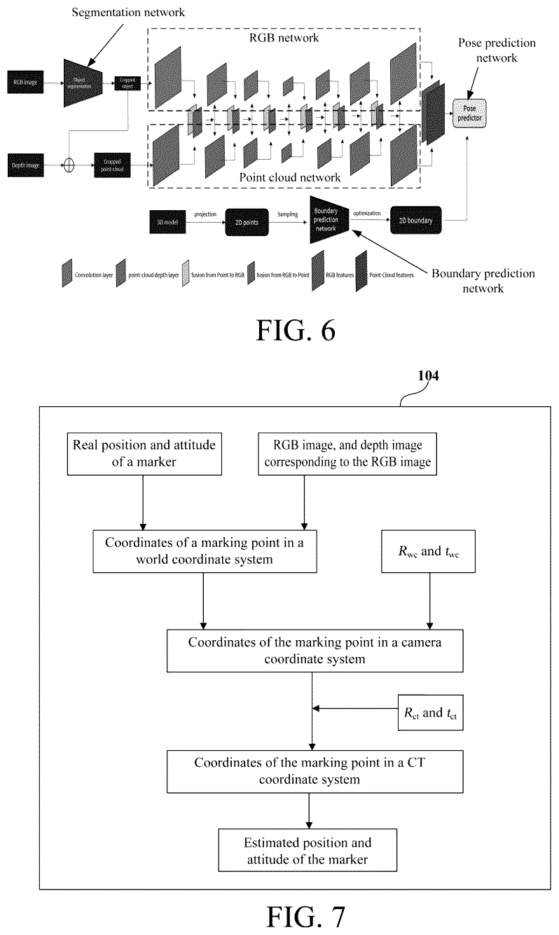

FIG. 5 is a schematic diagram of a working process of the positioning apparatus 103 according to an embodiment of the present application, and FIG. 6 is a schematic diagram of a working process of the trained network model. In the example of FIG. 6, the RGB image and the depth image are used as inputs, and an output is the estimated position and attitude of the marker 1011, etc. As shown in FIG. 5 and FIG. 6, the positioning apparatus 103 may separately process the RGB image of the marker 1011, and the depth image corresponding to the RGB image, that are acquired by the image acquisition apparatus 102, to obtain the position and the attitude of the marker 1011.

As shown in FIG. 5, the positioning apparatus 103 segments the RGB image by using the segmentation network, to separate a marker portion from the RGB image, so that information irrelevant to the marker 1011 can be removed, and an image background can be prevented from affecting detection of the marker 1011.

In the described embodiment, the marker portion may be used as an input to the RGB network, and the marker portion is processed by using the RBG network to extract an RGB feature of the marker part. Correspondingly, a point cloud part corresponding to the marker portion on the depth image corresponding to the RGB image may be used as an input to the point cloud network, and the point cloud network is used to process the point cloud portion, to extract a point cloud feature of the point cloud portion.

According to the described embodiment, the RBG feature and the point cloud feature of the marker portion are obtained, and the positioning apparatus 103 can estimate the position and the attitude of the marker based on the RBG feature and the point cloud feature.

In some embodiments, the positioning apparatus 103 may obtain a real position and attitude of the marker based on the RGB feature and the point cloud feature of the marker 1011 by using a pose prediction network.

The network structure of the trained network is not limited in the present application. In at least one example, the trained network may include a full flow bidirectional fusion network (FFB6D), in which the segmentation network, the RGR network, the point cloud network and the pose prediction network may be implemented by any implementable means. The trained network is described below using FIG. 5 and FIG. 6 as examples.

As shown in FIG. 5, the marker portion separated from the RGB image is convolved by the RGB network. In each convolutional layer, fusion from RGB to point cloud and fusion from point cloud to RGB are performed on a convolved feature and a point cloud depth layer in the corresponding point cloud network, to obtain a fused feature; then RGB information and point cloud information are extracted from the fused feature, and input to the RGB network and the point cloud network, respectively. The described fusion and extraction operations are continued in a next convolutional layer and point cloud depth layer, and so on, to obtain an RGB feature and a point cloud feature, the RBG feature being an output of the RGB network, and the point cloud feature being an output of the point cloud network. The quantity of fusion and extraction operations may be set based on specific requirements, and is not limited in the present application.

Therefore, RGB image information and corresponding depth image information in a captured image are fused, so that data complementation can be implemented, appearance and geometric features required for posture estimation are better presented, and accuracy of position and attitude estimation is improved.

In some embodiments, the positioning apparatus 103 may further predict a boundary of the marker 1011, and calculate the position and the posture of the marker 1011 based on the RGB feature, the point cloud feature, and the boundary of the marker 1011. Taking FIG. 5 as an example, the positioning apparatus 103 projects the depth image corresponding to the RGB image of the marker 1011 into two dimensions to obtain two-dimensional points of the depth image, samples the two-dimensional points, inputs the obtained sampling points into a boundary prediction network to predict the boundary of the marker 1011, and then obtains the real position and attitude of the marker 1011 based on the boundary of the marker 1011, the RGB feature and the point cloud feature, thereby further supplementing boundary prediction of the marker and improving accuracy of position and attitude estimation.

In the described embodiment, the network structure of the boundary prediction network is not limited, and the boundary prediction network may be implemented as a part of the trained network, or may be implemented by means of a separate deep learning network. In at least one example, the pose prediction network may use an iterative closest point (ICP) algorithm to estimate the real position and attitude, or may use other methods to estimate the real position and attitude; in this case, estimation of the real position and attitude of the marker 1011 is estimation of a position and an attitude of the marker 1011 in a world coordinate system.

In some embodiments, as shown in FIG. 5, the positioning apparatus 103 processes the real position and attitude of the marker 1011 by using a trained network model, and outputs the estimated position and attitude of the marker 1011. In at least one example, as shown in FIG. 1, the biopsy needle positioning system 100 includes a training apparatus 104, and the training apparatus 104 trains the network model used by the positioning apparatus 103 by using a plurality of RGB images of the marker at different angles, and depth images corresponding to the RGB images, that are acquired by the image acquisition apparatus 102, as well as real positions and attitudes of the marker at the different angles.

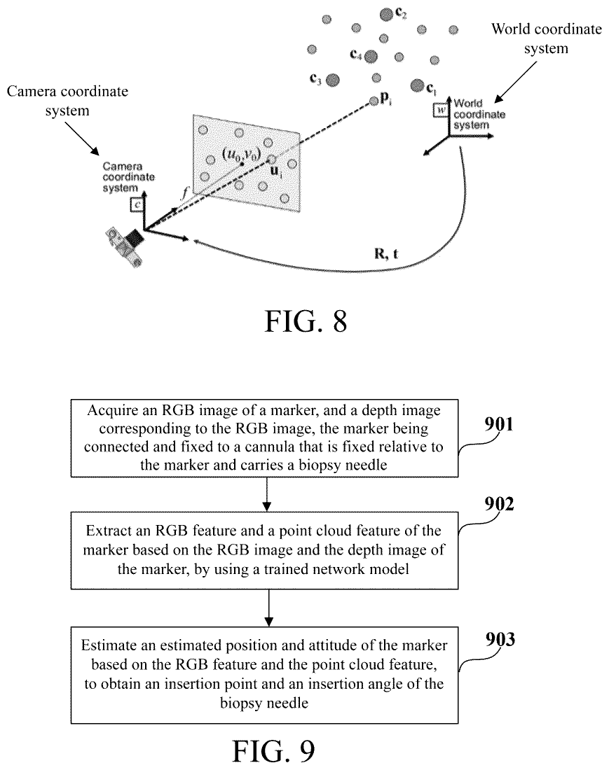

FIG. 7 is a schematic diagram of an action process of a training apparatus according to an embodiment of the present application, and FIG. 8 is a schematic diagram of conversion from a world coordinate system to a camera coordinate system. In some embodiments, for each of the RGB images of the marker 1011 at different angles, and each of the depth images corresponding to the RGB images, that are acquired by the image acquisition apparatus 102, the training apparatus 104 converts coordinates P_{world} of a plurality of marking points on the marker 1011 in the world coordinate system into coordinates P_{camera} in the camera coordinate system, and converts the coordinates P_{camera} of the plurality of marking points in the camera coordinate system into coordinates P_{ct} in a CT coordinate system, to obtain an estimated position and attitude of the marker at a current angle.

The marking points on the marker 1011 are located at different positions of the marker 1011 and represent a contour of the marker 1011, and the quantity and positions of the marking points may be set based on an actual requirement, as long as the contour of the marker 1011 can be completely represented. In addition, the coordinates P_{world} of the plurality of marking points in the world coordinate system may be obtained based on the RGB image and the depth image corresponding to the RGB image, and the real position and attitude of the marker 1011.

In some embodiments, the training apparatus 104 converts the coordinates P_{world} of the plurality of marking points in the world coordinate system into coordinates P_{camera} in the camera coordinate system by using the following formula:

$${P}_{camera} = {R}_{wc} \cdot {P}_{world} + {t}_{wc}$$

where R_{wc} and t_{wc} are external parameters of the image acquisition apparatus 102, R_{wc} being a rotation matrix and t_{wc} being a translation vector, which represent a position and a direction of the world coordinate system relative to the camera coordinate system, respectively.

In at least one example, R_{wc} is a 3*3 rotation matrix, t_{wc} is a 3*1 translation vector, and R_{wc} and t_{wc} may be obtained by using a perspective-n-point (PnP) algorithm; other algorithms may also be used to calculate R_{wc} and t_{wc} to obtain a conversion relationship from the world coordinate system to the camera coordinate system, so that a position and an attitude of the marker 1011 in the world coordinate system can be converted for representation in the camera coordinate system.

Taking FIG. 8 as an example, the perspective-n-point algorithm is used to calculate R_{wc} and t_{wc}. Coordinates of a point Pᵢ in the world coordinate system and coordinates of the point Pᵢ in a pixel coordinate system are known, and a rotation matrix and a translation vector of the camera coordinate system relative to the world coordinate system are calculated according to the formula, so that the coordinates in the world coordinate system can be converted into coordinates in the camera coordinate system.

In some embodiments, the training apparatus 104 converts the coordinates P_{camera} of the plurality of mark points in the camera coordinate system into coordinates P_{ct} in the CT coordinate system by using the following formula:

$${P}_{ct} = {R}_{ct} \cdot {P}_{camera} + {t}_{ct}$$

where R_{ct} is a rotation matrix, t_{ct} is a translation vector, which represent a position and a direction of the CT coordinate system relative to the camera coordinate system, respectively, and R_{ct} and t_{ct} are calculated by using a singular value decomposition (SVD) method; other algorithms may also be used.

In at least one example, the training apparatus 104 uses RGB images at a plurality of angles, and depth images corresponding to the RGB images, to calculate R_{ct} and t_{ct} by using the singular value decomposition method, and further uses a least square method to improve precision of the rotation matrix R_{ct} and the translation vector t_{ct}.

By means of calculating R_{ct} and t_{ct}, a conversion relationship from the camera coordinate system to the CT coordinate system is obtained, and the position and the attitude of the marker 1011 can be further converted for representation in the CT coordinate system.

In the described embodiment, it should be noted that the marker 1011 is fixed to the cannula 1012 and moves with the cannula 1012, so that the conversion relationship of the world coordinate system relative to the camera coordinate system, that is, values of R_{wc} and t_{wc}, changes with movement of the biopsy needle in RGB images at different angles, and depth images corresponding to the RGB images. For conversion between the camera coordinate system and the CT coordinate system, the values of R_{ct} and t_{ct} can be directly calculated based on relative positions of the image acquisition apparatus and a CT apparatus, and since the relative positions of the image acquisition apparatus 102 and the CT apparatus are fixed in most cases during biopsy, the values of R_{ct} and t_{ct} are also fixed in this case.

According to the described embodiment, the network model is trained, so that the network model obtains the coordinates of the plurality of marking points on the marker 1011 in the CT coordinate system, and outputs the estimated position and attitude of the marker 1011 based on the coordinates, that is, a representation of the marker 1011 in the CT coordinate system.

It should be noted that, in the described embodiment, only a training process for conversion of the marker 1011 between different coordinate systems is described, and the present application is not limited thereto. The training apparatus 104 may train all other parts in the network model, such as the segmentation network, the RGB network, the point cloud network, etc., thereby obtaining the trained network model.

In some embodiments, as shown in FIG. 5, the positioning apparatus can further calculate an estimated position and attitude of the biopsy needle whose position relative to the marker 1011 is fixed, based on the estimated position and attitude of the marker 1011, that is, calculate a position and an attitude of the biopsy needle in the CT coordinate system, and obtain the insertion point and angle of the biopsy needle based on the estimated position and attitude of the biopsy needle.

In some embodiments, as shown in FIG. 1, the biopsy needle positioning system 100 further includes a display apparatus 105, and the display apparatus 105 can display the insertion point and the insertion angle of the biopsy needle.

In some possible implementations, the display apparatus 105 can display the insertion point and the insertion angle of the biopsy needle. For example, when an operator operates the biopsy needle to move, the display apparatus 105 may display a green boundary box, or a boundary box of another color, to prompt the operator to operate based on a current insertion point and insertion angle, or based on other insertion points and insertion angles, or may use a prompt apparatus (not shown in the figure) to make a voice prompt, so as to inform the operator of the correct insertion point and insertion angle of the biopsy needle.

In at least one embodiment, the CT apparatus may be calibrated with respect to the relative positions of the image acquisition apparatus and the CT apparatus, so that a position and a direction between the CT coordinate system and the camera coordinate system will not cause inaccurate calculation of R_{ct} and t_{ct} due to changes in external factors. Therefore, the estimated position and posture of the marker 1011 can be further calibrated, so that the display apparatus 105 provides a more accurate biopsy needle insertion point and biopsy needle insertion angle. According to the described embodiment, biopsy costs are reduced, and work efficiency is improved.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

It can be learned from the described embodiment that the marker is fixed on the cannula carrying the biopsy needle, the real position and attitude of the marker are estimated based on the acquired RGB image corresponding to the marker and the depth image corresponding to the RGB image, and the estimated position and attitude of the marker are obtained by using the network model. Thus, the insertion point and angle of the biopsy needle are calculated by using the position and the attitude of the marker, the position of which is fixed relative to the biopsy needle, and the biopsy needle can be accurately positioned in real time without the need for a robotic arm and a re-calibration process, thereby reducing biopsy costs and improving work efficiency.

An embodiment of the present application further provides a biopsy needle positioning method; content the same as that of the foregoing embodiments will not be repeated.

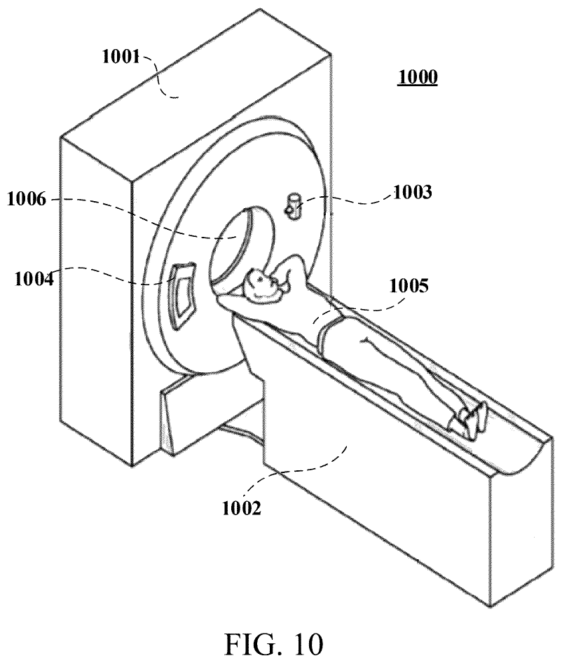

FIG. 9 is a schematic diagram of a biopsy needle positioning method according to an embodiment of the present application. As shown in FIG. 9, the biopsy needle positioning method includes the following steps:

901: Acquire an RGB image of a marker, and a depth image corresponding to the RGB image, the marker being connected and fixed to a cannula that is fixed relative to the marker and carries a biopsy needle.

902: Extract an RGB feature and a point cloud feature of the marker based on the RGB image and the depth image of the marker, by using a trained network model.

903: Estimate an estimated position and attitude of the marker based on the RGB feature and the point cloud feature, to obtain an insertion point and an insertion angle of the biopsy needle.

It should be noted that FIG. 9 merely schematically illustrates the embodiment of the present application, but the present application is not limited thereto. For example, the order of execution between operations may be appropriately adjusted. In addition, some other operations may be added or some operations may be omitted. Those skilled in the art may make appropriate variations based on the described content, rather than being limited to what is set forth in FIG. 9.

It is worth noting that only the steps related to the present application have been described above, but the present application is not limited thereto. The biopsy needle positioning method may further include other steps, and reference may be made to the related art for details of these steps.

An embodiment of the present application further provides a medical device system, which includes the biopsy needle positioning system 100 as described in the embodiment of the first aspect, the content of which is incorporated herein. The following provides an exemplary description of the medical device system. The medical device system described herein, that is, a device and a system that obtain medical imaging data, may be applied to various medical imaging modalities, including, but not limited to, computed tomography (CT) devices, positron emission tomography (PET)-CT, or any other suitable medical imaging device.

The system obtaining the medical imaging data may include the aforementioned medical imaging device, and may include a separate computer device connected to the medical imaging device, and may further include a computer device connected to an Internet cloud, the computer device being connected by means of the Internet to the medical imaging device or a memory for storing medical images. The imaging method may be independently or jointly implemented by the aforementioned medical imaging device, the computer device connected to the medical imaging device, and the computer device connected to the Internet cloud.

For example, the embodiments of the present application are described above in conjunction with an X-ray computed tomography (CT) device. Those skilled in the art would appreciate that the embodiments of the present application can also be applied to other medical imaging devices.

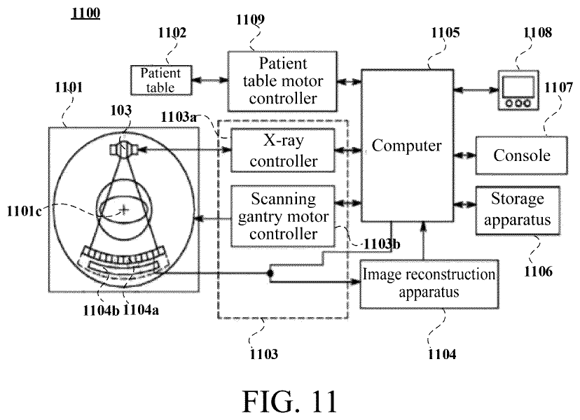

FIG. 10 is a schematic diagram of a CT imaging device according to an example of the present application, and schematically shows a CT imaging device 1000. Referring to FIG. 10, the CT imaging device 1000 includes a scanning gantry 1001 and a patient table 1002. The scanning gantry 1001 has an X-ray source 1003, the X-ray source 1003 projecting an X-ray beam toward a detector assembly or collimator 1004 on an opposite side of the scanning gantry 1001. A subject under examination 1005 can lie flat on the patient table 1002 and be moved into a scanning gantry opening 1006 along with the patient table 1002. Medical imaging data of the subject under examination 1005 can be obtained by means of scanning performed by the X-ray source 1003.

FIG. 11 is a schematic diagram of a CT imaging system according to an embodiment of the present application, and schematically shows a block diagram of a CT imaging system 1100. As shown in FIG. 11, the detector assembly 1104 includes a plurality of detector units 1104a and a data acquisition system (DAS) 1104b. The plurality of detector units 1104a sense a projected X-ray passing through a subject under examination 1105.

The DAS 1104b, based on sensing of the detector units 1104a, converts collected information into projection data for subsequent processing. During the scanning for acquiring the X-ray projection data, the scanning gantry 1101 and components mounted thereon rotate around a center of rotation 1101c.

The rotation of the scanning gantry 1101 and the operation of the X-ray source 1103 are controlled by a control mechanism 1103 of the CT imaging system 1100. The control mechanism 1103 includes an X-ray controller 1103a that provides power and a timing signal to the X-ray source 1103 and a scanning gantry motor controller 1103b that controls the rotational speed and position of the scanning gantry 1101. An image reconstruction apparatus 1104 receives the projection data from the DAS 1104b and performs image reconstruction. A reconstructed image is transmitted as an input to a computer 1105, and the computer 1105 stores the image in a mass storage apparatus 1106.

The computer 1105 also receives commands and scanning parameters from an operator through a console 1107. The console 1107 has an operator interface in a certain form, such as a keyboard, a mouse, a voice activated controller, or any other suitable input apparatus. An associated display 1108 allows the operator to observe a reconstructed image and other data from the computer 1105. The commands and parameters provided by the operator are used by the computer 1105 to provide control signals and information to the DAS 104b, the X-ray controller 1103a, and the scanning gantry motor controller 1103b. Additionally, the computer 1105 operates a patient table motor controller 1109, which controls the patient table 1102 to position the subject under examination 1105 and the scanning gantry 1101. In particular, the patient table 1102 moves the subject under examination 1105 to fully or partially pass through the scanning gantry opening 1106 in FIG. 11.

The device and system for acquiring medical image data (which may also be referred to as medical images or medical image data) according to the embodiments of the present application are schematically described above, but the present application is not limited thereto. The medical imaging device may be a CT device, a PET-CT, or any other suitable imaging device. A storage device may be located within the medical imaging device, in a server outside the medical imaging device, in an independent medical imaging storage system (such as a Picture Archiving and Communication System (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SAAS)”. The SAAS may exist between hospitals, between a hospital and an imaging center, or between a hospital and a third-party online diagnosis and treatment service provider.

The embodiments of the present application further provide a non-transitory computer-readable medium, having a computer program stored thereon, where the computer program has at least one code segment, and the at least one code segment is executable by a machine so that the machine performs steps of the method according to the foregoing embodiments. Since the specific implementation of the method has been described in the foregoing embodiments, the contents of which are incorporated herein, no further description is provided herein.

The above method of the present application may be implemented by hardware, or may be implemented by hardware in combination with software. The present application relates to such a computer-readable program that when executed by a logic component, the program enables the logic component to implement the constituent components described above, or enables the logic component to implement various methods or steps as described above. The present application further relates to a storage medium for storing the above program, such as a hard disk, a disk, an optical disk, a DVD, a flash memory, etc.

The method described with reference to the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the embodiments are clear according to the detailed description. Therefore, the appended claims are intended to cover all these features and advantages that fall within the true spirit and scope of these embodiments. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the embodiments.

1. A biopsy needle positioning system, comprising:
a marker assembly including a marker and a cannula that is fixed relative to the marker and carries a biopsy needle;
an image acquisition apparatus, the image acquisition apparatus being mounted at a position at which the marker can be photographed, and configured to acquire an RGB image of the marker and a depth image corresponding to the RGB image; and
a positioning apparatus, which extracts an RGB feature and a point cloud feature of the marker based on the RGB image and the depth image of the marker by using a trained network model, and estimates an estimated position and attitude of the marker based on the RGB feature and the point cloud feature, to obtain an insertion point and an insertion angle of the biopsy needle.

2. The system according to claim 1, wherein the marker assembly further comprises a bracket connected to one end of the cannula, the marker being mounted on the bracket.

3. The system according to claim 2, wherein the bracket is L-shaped.

4. The system according to claim 2, wherein the marker is a cube, a sphere, or a cone.

5. The system according to claim 1, wherein the positioning apparatus further:
segments the RGB image by using a segmentation network, to separate a marker portion from the RGB image;
extracts an RGB feature of the marker portion by using the marker portion as an input to an RGB network;
extracts a point cloud feature of a point cloud portion corresponding to the marker portion on the depth image by using the point cloud portion as an input to a point cloud network; and
obtains a real position and attitude of the marker based on the RGB feature and the point cloud feature.

6. The system according to claim 5, wherein the positioning apparatus further:
projects the depth image into two dimensions, samples obtained two-dimensional points, and predicts a boundary of the marker by using a boundary prediction network; and
obtains the real position and attitude of the marker based on the RGB feature, the point cloud feature, and the boundary of the marker.

7. The system according to claim 1, further including a training apparatus, configured to train the network model used by the positioning apparatus by using a plurality of RGB images of the marker at different angles and depth images corresponding to the RGB images that are acquired by the image acquisition apparatus, and real positions and attitudes of the marker at the different angles.

8. The system according to claim 7, wherein for each of the RGB images and the depth images corresponding to the RGB images, the training apparatus converts coordinates P_{world} of a plurality of marking points on the marker in a world coordinate system into coordinates P_{camera} in a camera coordinate system, and converts the coordinates P_{camera} of the plurality of marking points in the camera coordinate system into coordinates P_{ct} in a CT coordinate system, to obtain an estimated position and attitude of the marker at a current angle.

9. The system according to claim 8, wherein the training apparatus converts the coordinates of the plurality of marking points in the world coordinate system into coordinates in the camera coordinate system using external parameters of the image acquisition apparatus which represent a position and a direction of the camera coordinate system relative to the world coordinate system.

10. The system according to claim 8, wherein the training apparatus converts the coordinates of the plurality of marking points in the camera coordinate system into coordinates in the CT coordinate system, using a position and a direction of the CT coordinate system relative to the camera coordinate system being calculated by means of a singular value decomposition method.

11. The system according to claim 8, wherein the plurality of marking points are located at different positions of the marker, and the plurality of marking points represent a contour of the marker.

12. The system according to claim 1, wherein the system further includes a display apparatus to display an insertion point and an insertion angle of the biopsy needle.

13. A biopsy needle positioning method, characterized in that the method comprises:
acquiring an RGB image of a marker and a depth image corresponding to the RGB image, wherein the marker is connected and fixed to a cannula that is fixed relative to the marker and carries a biopsy needle;
extracting an RGB feature and a point cloud feature of the marker based on the RGB image and the depth image of the marker by using a trained network model; and
estimating an estimated position and attitude of the marker based on the RGB feature and the point cloud feature, to obtain an insertion point and angle of the biopsy needle.
